(19) United States
(12) Patent Application Publication
(10) Pub. No.: US 2020/0189989 A1
(43) Pub. Date: Jun. 18, 2020

(54) APPLICATION OF SYRINGIC ACID IN PROMOTING NITRIFICATION INHIBITION ACTIVITY OF DECANEDIOL

(71) Applicant: Institute of Soil Science, Chinese Academy of Sciences, Nanjing (CN)

(72) Inventors: Weiming Shi, Nanjing (CN); Xiaonan Zhang, Nanjing (CN); Yufang Lu, Nanjing (CN)

(21) Appl. No.: 16/412,802

(22) Filed: May 15, 2019

(30) Foreign Application Priority Data
Dec. 13, 2018 (CN) 201811524991.8

(51) Int. Cl. C05G 3/08

(52) U.S. Cl. CPC C05G 3/08; C07C 65/21

(57) ABSTRACT

An application of syringic acid in promoting the inhibition effect of decanediol on the nitrosification activity of nitrosomonas is provided. By inhibiting the activity of a nitrosomonas, the combined use of syringic acid and 1,9-decanediol can more efficiently inhibit a nitrification process in soil and at the plant rhizosphere, increase the nitrogen use efficiency, and reduce leaching of nitrate nitrogen, thereby reducing the loss of nitrogen and discharge of the greenhouse gas nitrous oxide in a denitrification process. The addition of syringic acid in the present invention can reduce the concentration at which 1,9-decanediol functions under the equivalent condition of nitrification inhibition activity, and thus save the relative input cost.

### CROSS-REFERENCE TO RELATED APPLICATION

This application claims priority to Chinese Patent Application No. 201811524991.8, filed Dec. 13, 2018. The above-mentioned patent application is incorporated herein by reference in its entirety.

### TECHNICAL FIELD

The present invention relates to nitrification inhibitors, and more particularly, relates to an application of syringic acid as a nitrification inhibitor.

### BACKGROUND

NH₄⁺ produced by mineralization of organic matters in the soil and NH₄⁺ added by fertilizers are easily adsorbed by negatively-charged functional groups of clay and soil organics, and thus the loss of them is limited. However, NH₄⁺ is easily converted to NO₃⁻ by nitrification microbes in the soil through nitrification. As compared with NH₄⁺, the NO₃⁻ has weaker adsorption in the soil, and thus is easily lost through leaching, runoff and the like pathways, which results in direct loss of nitrogen, and meanwhile seriously pollutes underground water and surface water, causing environmental problems such as non-point source pollution; and the acting of denitrification in the soil would convert NO₃⁻ into N₂ and N₂O to be lost into the environment, causing further loss of nitrogen from the soil. Meanwhile, N₂O, as a greenhouse gas, has a warming potential which is 298 times greater than CO₂, is closely related to global warming and tropospheric ozone depletion, and has an increasingly serious potential damage to the environment. One of the important sources of it is the nitrification and denitrification process of nitrogen in a farmland ecosystem.

In an agroecological system, due to the nitrogen loss caused by a direct or indirect action of nitrification, the nitrogen fertilizer absorbed and utilized through conversion by a plant does not exceed 30%, and thus the nitrogen use efficiency of the crop is relatively lower. In order to reduce the nitrogen loss of nitrification, in the production practice, people often adopt some nitrogen fertilizer management measures and apply a controlled-release fertilizer, but the labor and agricultural capital costs increased accordingly cannot be underestimated, and the effect is not satisfactory. Furthermore, some synthetic nitrification inhibitors are often used in agricultural production together with nitrogen fertilizers, which can effectively control nitrogen loss of soil and improve the nitrogen use efficiency of a crop. However, these nitrification inhibitors have limitations such as a having a high price, having an unstable performance, being cumbersome to use, being liable to cause environmental pollution, and affecting the biodiversity of the natural ecosystem.

The biological nitrification inhibitor is a compound having a nitrification inhibiting ability, which is synthesized or secreted by a plant. It is derived from a plant extract or a root exudate, and thus has the advantages of being easily accessible and environmentally friendly as compared with a synthetic nitrification inhibitor. It has been reported in a literature that a specific nitrification inhibiting substance 1,9-decanediol is existed in the root exudate of rice, which has a good nitrification inhibition effect. Moreover, the field and pot experiments show that 1,9-decanediol can inhibit the abundance of an amoA gene of AOB and AOA at the same time, and thus inhibit the nitrification in soil, which is not available by the synthetic nitrification inhibitor. However, it is found through the field and pot experiments that, the concentration at which 1,9-decanediol functions is relatively large, and the synthesis cost of 1,9-decanediol is relatively high, which makes it be difficult to practically popularize and apply 1,9-decanediol. However, the root exudate of a plant is a kind of complex mixture which contains a variety of active ingredients therein. The ultimate inhibitory activity exhibited by a plant in the natural environment is not only related to a monomeric substance having an inhibitory potential, but also related to the interaction between various substances, which is an important factor that affects the inhibitory activity of a plant secreta.

Therefore, it would be desirable to provide one or more natural compounds to be co-administered with 1,9-decanediol in such a manner that the concentration at which 1,9-decanediol functions is lowered without affecting the nitrification inhibitory activity, thereby reducing the use cost.

### SUMMARY

To achieve the above purposes and solve the technical defects in the art, an application of syringic acid is provided for promoting a nitrification inhibition activity of 1,9-decanediol. By inhibiting the activity of a nitrosomonas, the combined use of syringic acid and 1,9-decanediol can more efficiently inhibit a nitrification process in soil and at the plant rhizosphere, increase the nitrogen use efficiency, and reduce leaching of nitrate nitrogen, thereby reducing the loss of nitrogen and discharge of the greenhouse gas nitrous oxide in a denitrification process. The addition of syringic acid in the present invention can reduce the concentration at which 1,9-decanediol functions under the equivalent condition of nitrification inhibition activity, and thus save the relative input cost, and meanwhile syringic acid and 1,9-decanediol are both plant-derived hydrophobic substances having stable chemical structures, which improves the problems of the traditional nitrification inhibitor that it is easy to loss, has a short-term effectiveness and may cause environmental pollution, and enhances the nitrification inhibition efficiency.

An application of syringic acid in promoting the inhibition effect of 1,9-decanediol on the nitrosification activity of Nitrosomonas is provided in one embodiment of the present invention.

In one aspect, the nitrosomonas is Nitrosomonas europaea (NBRC 14298) and Nitrosomonas stercoris (NBRC 110753).

In another embodiment of the invention, an application of syringic acid is provided in preparation of a formulation for promoting the inhibition effect of 1,9-decanediol on the nitrosification activity of nitrosomonas.

In another embodiment of the invention, an application of syringic acid is provided in preparation of a composition for reducing nitrogen loss in farmland.

The root exudates of 19 rice varieties are collected, concentrated, and determined for their nitrosification inhibition activities by using Nitrosomonas europaea. The varieties with significant effects are further screened out from them, to select a variety of which the root exudate has a significant inhibitory effect and a variety of which the root exudate has an effect opposite to the former, and then they are subjected to GC/MS identification for components thereof. By component comparison of the two varieties, syringic acid (4-Hydroxy 3,5-dimethoxybenzoic acid, with the molecular weight of 198.18) is identified in the root exudate of the rice variety Wuyunjing 7 having a significant inhibitory effect. This compound has no significant inhibitory activity to the Nitrosomonas, but it has a significant promoting effect in the inhibition of 1,9-decanediol to the nitrosification activity of the Nitrosomonas, and its effect concentration is 10-500 μg·mL⁻¹.

Embodiments of the present invention achieve the following beneficial effects. (1) Syringic acid is applied in promoting the nitrification inhibitory activity of 1,9-decanediol, and syringic acid at a low dose can efficiently promote the inhibition effect of 1,9-decanediol on the Nitrosomonas, reduce nitrogen loss and environmental problems caused by the nitrification process in soil of farmland. (2) There is no standard of 1,9-decanediol on the market, and thus it is a synthetic product of our company, which has a relatively higher synthesis cost at about 600 yuan/gram, while syringic acid is a chemical product sold on the market at a price of about 1.5 yuan/gram, and the addition of syringic acid can promote the nitrification inhibitory activity of 1,9-decanediol and can solve problems of 1,9-decanediol such as a large effect dose concentration and a high synthesis cost, thereby reducing the relative input cost. (3) Both syringic acid and 1,9-decanediol are hydrophobic compounds having stable chemical structures, which are not easy to lose in soil and water, and thus can ensure the stability and high efficiency of application. (4) Both syringic acid and 1,9-decanediol are plant-derived “green” organic substances, which can solve the problems of the conventional chemically-synthesized nitrification inhibitor that it has a short-term effectiveness, a poor effect and is easy to cause pollution, while providing new methods and ideas for constructing a bio-ecological technology that effectively inhibits the nitrification process of a farmland ecosystem and improves the nitrogen use efficiency.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitutes a part of this specification, illustrate one or more embodiments of the invention and, together with the general description given above and the detailed description given below, explain the one or more embodiments of the invention.

FIG. 1 is a graphical plot showing a GC/MS analysis spectrum of the components in the root exudate of the rice variety Wuyunjing 7 according to one embodiment of the present invention, showing that 1,9-decanediol is a substance which has a peak at 20.04 min, and syringic acid is a substance which has a peak at 27.44 min.

FIG. 2 is a graphical bar chart showing the effect of syringic acid on the nitrification inhibition activity of 1,9-decanediol to the Nitrosomonas europaea (mean±SE, n=3) according to embodiments of the invention, where different lowercase letters indicate significant differences between groups (P<0.05).

FIG. 3 is a graphical bar chart showing the effect of syringic acid on the nitrification inhibition activity of 1,9-decanediol to the Nitrosomonas stercoris (mean±SE, n=3) according to embodiments of the invention, where different lowercase letters indicate significant differences between groups (P<0.05).

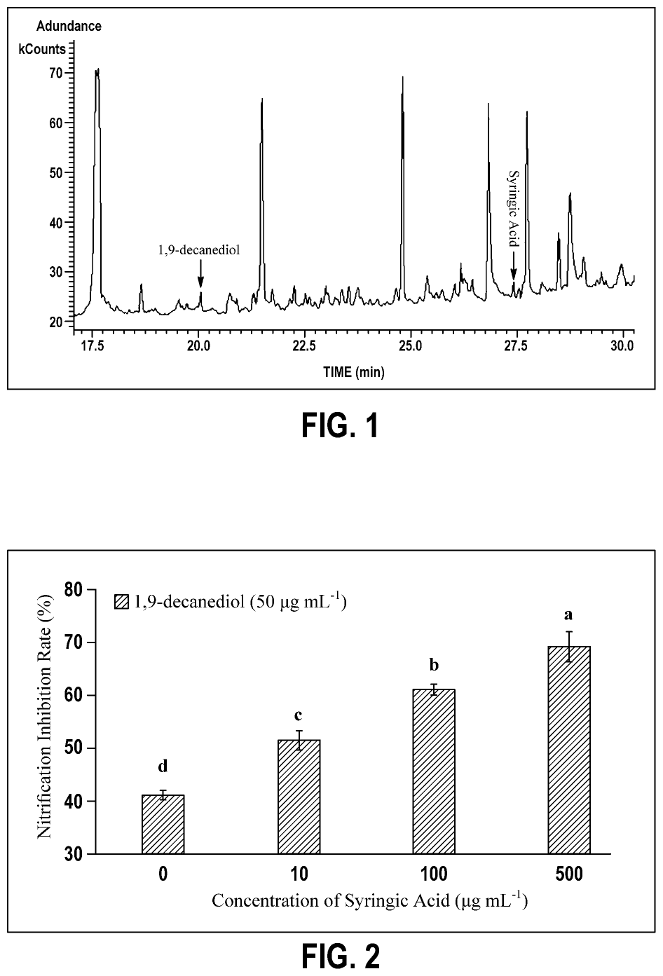

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to accompanying drawings and specific implementations.

Embodiment 1: The Effect of Syringic Acid on the Nitrification Inhibition Activity of 1,9-Decanediol to the Nitrosomonas europaea

### 1.1 Experimental Design

Standard: a syringic acid standard was purchased from Sigma-Aldrich (St. Louis, Mo., USA), and the solid powder thereof was weighed and dissolved in DMSO. A 1,9-decanediol standard was purchased from WuXi AppTec, and the solid powder thereof was dissolved in DMSO while frozen over the dry ice (the 1,9-decanediol standard was a viscous liquid substance under normal temperature).

Microbial strains: Nitrosomonas europaea (NBRC 14298) was purchased from Biological Resource Center, NITE, Japan.

Microbial culture medium: a HEPES medium, which contained 2.5 g of (NH₄)₂SO₄, 0.5 g of KH₂PO₄, 11.92 g of HEPES, 0.5 g of NaHCO₃, 100 mg of MgSO_{4.}7H₂O, 5 mg of CaCl₂.2H₂O, and 75 mg of Fe-EDTA per 1 L liquid medium, at pH 7.8-8.0.

Culture of microorganisms: Nitrosomonas europaea was inoculated in the HEPES medium and cultured at 30° C. and 200 rpm under a dark condition (aerobic), and the Nitrosomonas europaea enters a stable period 7-9 days after transfer each time.

Nitrification activity inhibition experiment: The bacterial solution cultured for 7 days was collected, centrifuged at 5000 g for 20 min, and resuspended in a fresh and sterile HEPES medium until the OD600 reached about 1.0, with a concentration multiple of 40-50 times. Taken was a 1.5 mL sterile centrifuge tube, and it was sequentially added with 195 μL of sterile water, 5 μL of different concentrations of solutions of syringic acid and 1,9-decanediol in DMSO, 100 μL of the fresh and sterile HEPES medium, and 200 μL of a resuspended bacterial solution, and then cultured in a water bath at 25° C. under a dark condition for 2 hours. Then the mixed system was added with 20 μL of 0.1M Allylthiourea to terminate the nitrosification. 200-400 pL of the reacted mixture was added into a 10 mL colorimetric tube, diluted to about 5 mL with deionized water, added with 1 mL of a sulfanilic acid solution, shaken well and then subjected to standing for 2-8 min, then added with 1 mL of a hydrochloric acid N-(1-naphthyl)-ethylenediamine solution, shaken well, diluted to 10 mL with water to obtain a constant volume. Deionized water was used as a reference, and the absorbances were determined at a wavelength of 540 nm. The reticle of NO₂⁻ was made in the same manner to quantify the NO₂⁻ generated in the sample system, and the sample inhibition rate was calculated by the following formula. This determination method was an improved Griess method, which could be referred to the national standard “determination of nitrite in the wet precipitation -N-(1-naphthyl)-1,2-diaminoethane spectrophotometry”.

Nitrosification inhibition rate (%)=(1-the produced amount of NO₂⁻ of the sample/the produced amount of NO₂⁻ of a blank)×100%

### 1.2. Experimental Result

The experimental result as shown in FIG. 2 was that syringic acid had a significant synergistic effect on the nitrification inhibition activity of 1,9-decanediol. When 1,9-decanediol was added alone, its inhibition rate for the nitrosification process of Nitrosomonas europaea was 41.2%, and after syringic acid each at the concentrations of 10, 100 and 500 μ·mL⁻¹ syringic acid was added on this basis, the nitrification inhibition effect of 1,9-decanediol was significantly improved in such a manner that the nitrification inhibition rates were increased to 51.7%, 61.1% and 69.2%, respectively.

Embodiment 2: The effect of syringic acid on the nitrification inhibition activity of 1,9-decanediol to the Nitrosomonas stercoris 1.1 Experimental Design

The selection and formulation of the standard were the same as those of Embodiment 1.

Microbial Strains: Nitrosomonas stercoris (NBRC 110753) was purchased from Biological Resource Center, NITE, Japan.

The microbial culture medium and microbial culture were the same as those of Embodiment 1.

Nitrification activity inhibition experiment: The bacterial solution of Nitrosomonas stercoris cultured for 7 days was collected, centrifuged at 5000 g for 20 min, and resuspended in a fresh and sterile HEPES medium until the OD600 reached about 1.0, with a concentration multiple of 40-50 times. Taken was a 1.5 mL sterile centrifuge tube, and it was sequentially added with 195 μL of sterile water, 5 μL of different concentrations of solutions of syringic acid and 1,9-decanediol in DMSO, 100 μL of the fresh and sterile HEPES medium, and 200 μL of a resuspended bacterial solution, and then cultured in a water bath at 25° C. under a dark condition for 2 hours. Then the mixed system was added with 20 μL of 0.1M Allylthiourea to terminate the nitrosification. The determination method for the NO₂⁻ generated in the system after the reaction was the same as that of Embodiment 1.

### 1.2. Experimental Result

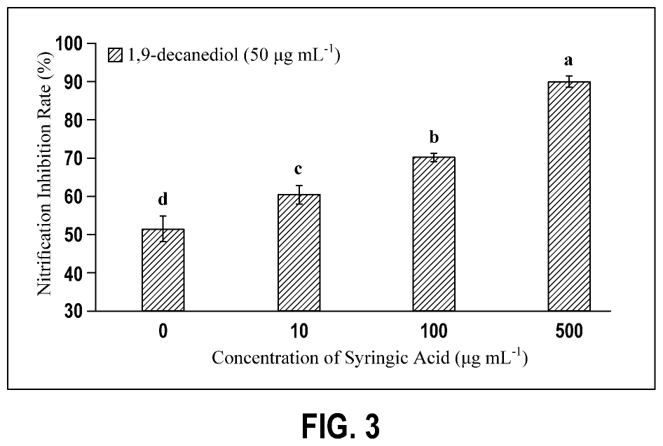

The experimental result as shown in FIG. 3 was that, syringic acid also promoted the inhibition effect of 1,9-decanediol on Nitrosomonas stercoris, where when 10, 100 and 500 μg·mL⁻¹ of syringic acid were added, the nitrification inhibition effect of 1,9-decanediol was significantly increased, with the inhibition rate increased from 51.5% to 60.5%, 70.2% and 89.9% accordingly. Therefore, syringic acid has a good application prospect in promoting the inhibition activity of 1,9-decanediol on the Nitrosomonas.

The embodiments described above are only descriptions of preferred embodiments of the present invention, and do not intended to limit the scope of the present invention. Various variations and modifications can be made to the technical solution of the present invention by those of ordinary skills in the art, without departing from the design and spirit of the present invention. The variations and modifications should all fall within the claimed scope defined by the claims of the present invention.

What is claimed is:

1. An application of syringic acid to promote an inhibition effect of 1,9-decanediol on the nitrosification activity of a nitrosomonas.

2. The application of syringic acid of claim 1, wherein the nitrosomonas is Nitrosomonas europaea (NBRC 14298) and Nitrosomonas stercoris (NBRC 110753).

3. The application of syringic acid of claim 1, wherein an addition concentration of syringic acid is 10-500 μg·mL⁻¹.

4. An application of syringic acid to prepare a formulation for promoting an inhibition effect of 1,9-decanediol on the nitrosification activity of a nitrosomonas.

5. An application of syringic acid to prepare a composition for reducing nitrogen loss in farmland.
